(19) United States
(12) Patent Application Publication
(10) Pub. No.: US 2007/0275948 A1
(43) Pub. Date: Nov. 29, 2007

(54) 7-[4-(4-Chlorobenzyloxy)Benzenesulfonyl]-8-Methoxy-3-Methyl-2,3,4,5-Tetrahydro-1h-3-Benzazepinium Maleate Or Tosylate As antipyschotics

(76) Inventor: David Clapham, Harlow (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/580,640

(22) PCT Filed: Nov. 25, 2004
(86) PCT No.: PCT/EP04/13416

§ 371 Date: May 25, 2006

(30) Foreign Application Priority Data
Nov. 28, 2003 (GB) 0327740.7

(51) Int. Cl. A61K 31/55; A61P 25/18; C07D 223/16

(52) U.S. Cl. 514/217.01; 540/594

(57) ABSTRACT

The present invention relates to novel salts of 7-[4-(4-chlorobenzyloxy)benzenesulfonyl]-8-methoxy-3-methyl-2,3,4,5-tetrahydro-1H-3-benzazepine and a pharmaceutically acceptable solvate thereof, pharmaceutical formulations, processes for their preparation, and their use in medicine. The chemical entities are useful in therapy, in particular as antipsychotic agents.

The present invention relates to novel salts of 7-[4-(4-chlorobenzyloxy)benzenesulfonyl]-8-methoxy-3-methyl-2,3,4,5-tetrahydro-1H-3-benzazepine and a pharmaceutically acceptable solvate thereof, pharmaceutical formulations, processes for their preparation, and their use in medicine.

The structure of 7-[4-(4-chlorobenzyloxy)benzenesulfonyl]-8-methoxy-3-methyl-2,3,4,5-tetrahydro-1H-3-benzazepine is indicated below as the compound of formula (I):

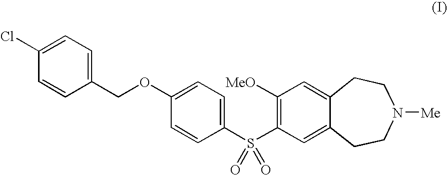

The compound of formula (I) can be prepared by the reaction of 7-(4-fluoro-benzenesulfonyl)-8-methoxy-3-methyl-2,3,4,5-tetrahydro-1H-3-benzazepine with 4-chlorobenzyl alcohol in a suitable solvent, for example, tetrahydrofuran, in the presence of a base, for example, potassium tert-butoxide.

The hydrochloride salt of 7-[4-(4-chlorobenzyloxy)benzenesulfonyl)-8-methoxy-3-methyl-2,3,4,5-tetrahydro-1H-3-benzazepine can be prepared by treatment of 7-[4-(4-chlorobenzyloxy)benzenesulfonyl)-8-methoxy-3-methyl-2,3,4,5-tetrahydro-1H-3-benzazepine free base with ethereal hydrogen chloride and crystallisation from ethanol.

The compound of formula (I) and its pharmaceutically acceptable salts and solvates are reported in WO 03/099786 to be useful as antipsychotic agents for example in the treatment of schizophrenia, schizo-affective disorders and schizophreniform diseases and other disorders such as psychotic depression (which term includes bipolar depression, unipolar depression, single or recurrent major depressive episodes with or without psychotic features, catatonic features, melancholic features, atypical features or postpartum onset, seasonal affective disorder and dysthymia, depressive disorders resulting from a general medical condition including, but not limited to, myocardial infarction, diabetes, miscarriage or abortion), anxiety disorders (which includes generalised anxiety and social anxiety disorder), mania, acute mania, paranoid and delusional disorders.

For use in medicine there exists a need for a compound to be prepared in a form suitable for ease of isolation in large scale manufacture and ease of formulating into an acceptable product for administration to patients. It is difficult to predict the physical characteristics of any particular salt of a compound and small, but significant, differences in physical properties may equate to large savings in the manufacture and formulation of a pharmaceutical product containing a compound.

The compound of formula (I) as a free base (hereinafter also referred to as “the free base”) exists in multiple forms and all of those tested have been observed to be hygroscopic. The compound of formula (I) as the hydrochloride salt also exists in multiple forms and all of those tested have also been observed to be hygroscopic. This hygroscopicity affects the ease of handling of the compound of formula (I) under ambient conditions. The hygroscopicity affects the ability to accurately weigh the material, therefore control of atmospheric conditions, for example by use of a glove-box, are necessary to prevent the compound of formula (I) from absorbing water during procedures such as weighing out and formulation. It will be appreciated that it is vitally important to ensure consistent and accurate weight of active compound in a pharmaceutical composition.

The present invention provides a novel salt of 7-[4-(4-chlorobenzyloxy)benzenesulfonyl)-8-methoxy-3-methyl-2,3,4,5-tetrahydro-1H-3-benzazepine selected from maleate and p-toluenesulfonate (hereinafter also referred to as “the maleate” and “the tosylate” respectively), which may be used as an alternative to either the free base or the hydrochloride salt of the compound of formula (I) for therapeutic administration or as an intermediate in the preparation of other salts. The invention also provides novel methods of preparation of these novel salts of the compound of formula (I) which are suitable for commercial use.

The maleate and tosylate salts of 7-[4-(4-chlorobenzyloxy)benzenesulfonyl]-8-methoxy-3-methyl-2,3,4,5-tetrahydro-1H-3-benzazepine are particularly suited to large scale preparation. Such processes may be for example efficient processes, economic processes or reproducible processes.

The maleate and tosylate salts of 7-[4-(4-chlorobenzyloxy)benzenesulfonyl]-8-methoxy-3-methyl-2,3,4,5-tetrahydro-1H-3-benzazepine have improved stability over the free base and over the hydrochloride salt of 7-[4-(4-chlorobenzyloxy)benzenesulfonyl]-8-methoxy-3-methyl-2,3,4,5-tetrahydro-1H-3-benzazepine, particularly with respect to hygroscopicity.

The maleate and tosylate salts of the compound of formula (I) may be easier to manufacture than the free base and the hydrochloride salt and may be advantageous in the preparation of certain pharmaceutical compositions.

Therefore, as a first aspect of the present invention there is provided one or more chemical entities selected from 7-[4-(4-chlorobenzyloxy)benzenesulfonyl]-8-methoxy-3-methyl-2,3,4,5-tetrahydro-1H-3-benzazepinium maleate and a pharmaceutically acceptable solvate thereof.

In another aspect of the present invention there is provided one or more chemical entities selected from 7-[4-(4-chlorobenzyloxy)benzenesulfonyl]-8-methoxy-3-methyl-2,3,4,5-tetrahydro-1H-3-benzazepinium tosylate and a pharmaceutically acceptable solvate thereof.

In a further aspect of the present invention there is provided 7-[4-(4-chlorobenzyloxy)-benzenesulfonyl]-8-methoxy-3-methyl-2,3,4,5-tetrahydro-1H-3-benzazepinium maleate in which the ratio of 7-[4-(4-chlorobenzyloxy)benzenesulfonyl]-8-methoxy-3-methyl-2,3,4,5-tetrahydro-1H-3-benzazepine to maleic acid (by mole) is 1:1.

In another aspect of the present invention there is provided 7-[4-(4-chlorobenzyloxy)-benzenesulfonyl]-8-methoxy-3-methyl-2,3,4,5-tetrahydro-1H-3-benzazepinium tosylate in which the ratio of 7-[4-(4-chlorobenzyloxy)benzenesulfonyl]-8-methoxy-3-methyl-2,3,4,5-tetrahydro-1H-3-benzazepine to p-toluenesulfonic acid (by mole) is 1:1.

Depending on the solvent from which the maleate is recovered, the maleate is obtained as a solvate and such a solvate also forms one aspect of the present invention. The solvate may be a pharmaceutically acceptable solvate. Suitable solvates include hydrates, such as dihydrate, and acetic acid solvates.

Depending on the solvent from which the tosylate is recovered, the tosylate may be obtained as a solvate and such a solvate also forms one aspect of the present invention. The solvate may be a pharmaceutically acceptable solvate. Suitable solvates may include hydrates.

Alternatively, the maleate and tosylate are each obtained as anhydrates. The anhydrate may contain less than 2% water, for example less than 1% water. The maleate and tosylate anhydrates independently demonstrate particular stability with respect to hygroscopicity and loss of water. Furthermore, the maleate and tosylate anhydrates demonstrate reversible changes when exposed to very high humidity.

In a further aspect there is provided one or more chemical entities selected from the maleate and a pharmaceutically acceptable solvate thereof in isolated form. In a yet further aspect there is provided one or more chemical entities selected from the maleate and a pharmaceutically acceptable solvate thereof which is substantially free of alternative salts, alternative solvates or free base of a compound of formula (I) or other impurity.

In another aspect there is provided one or more chemical entities selected from the tosylate and a pharmaceutically acceptable solvate thereof in isolated form. In a further aspect there is provided one or more chemical entities selected from the tosylate and a pharmaceutically acceptable solvate thereof which is substantially free of alternative salts, alternative solvates or free base of a compound of formula (I) or other impurity.

By “substantially free of alternative salts, alternative solvates or free base of a compound of formula (I) or other impurity” is meant containing less than 10%, for example less than 5%, such as less than 2%, of alternative salts, alternative solvates or free base of a compound of formula (I) or other impurity. The term “other impurity” includes any compound other than the compound of formula (I).

The maleate and a pharmaceutically acceptable solvate thereof may each be obtained in a non-crystalline or crystalline form. The tosylate and a pharmaceutically acceptable solvate thereof may each be obtained in a non-crystalline or crystalline form.

In a still further aspect there is provided one or more chemical entities selected from the maleate and a pharmaceutically acceptable solvate thereof in at least one polymorphic form(s). In another aspect there is provided one or more chemical entities selected from the tosylate and a pharmaceutically acceptable solvate thereof in at least one polymorphic form(s).

We have discovered that crystalline 7-[4-(4-chlorobenzyloxy)benzenesulfonyl]-8-methoxy-3-methyl-2,3,4,5-tetrahydro-1H-3-benzazepinium maleate in which the ratio of 7-[4-(4-chlorobenzyloxy)benzenesulfonyl]-8-methoxy-3-methyl-2,3,4,5-tetrahydro-1H-3-benzazepine to maleic acid (by mole) is 1:1, independently exists in at least one polymorphic form.

We have discovered that crystalline 7-[4-(4-chlorobenzyloxy)benzenesulfonyl]-8-methoxy-3-methyl-2,3,4,5-tetrahydro-1H-3-benzazepinium tosylate, in which the ratio of 7-[4-(4-chlorobenzyloxy)benzenesulfonyl]-8-methoxy-3-methyl-2,3,4,5-tetrahydro-1H-3-benzazepinium to p-toluenesulfonic acid (by mole) is 1:1, independently exists in at least one polymorphic form.

We have discovered that crystalline 7-[4-(4-chlorobenzyloxy)benzenesulfonyl]-8-methoxy-3-methyl-2,3,4,5-tetrahydro-1H-3-benzazepinium maleate, dihydrate in which the ratio of 7-[4-(4-chlorobenzyloxy)benzenesulfonyl]-8-methoxy-3-methyl-2,3,4,5-tetrahydro-1H-3-benzazepine to maleic acid (by mole) is 1:1, independently exists in at least one polymorphic form.

We have discovered that crystalline 7-[4-(4-chlorobenzyloxy)benzenesulfonyl]-8-methoxy-3-methyl-2,3,4,5-tetrahydro-1H-3-benzazepinium maleate, acetic acid solvate in which the ratio of 7-[4-(4-chlorobenzyloxy)benzenesulfonyl]-8-methoxy-3-methyl-2,3,4,5-tetrahydro-1H-3-benzazepine to maleic acid (by mole) is 1:1, independently exists in at least one polymorphic form.

Accordingly a further aspect of the invention provides

7-[4-(4-chlorobenzyloxy)benzenesulfonyl]-8-methoxy-3-methyl-2,3,4,5-tetrahydro-1H-3-benzazepinium(1:1) maleate having an X-Ray powder diffraction (XRPD) pattern with signals substantially as listed in Table 1.

Accordingly a further aspect of the invention provides

7-[4-(4-chlorobenzyloxy)benzenesulfonyl]-8-methoxy-3-methyl-2,3,4,5-tetrahydro-1H-3-benzazepinium(1:1) tosylate having an XRPD pattern with signals substantially as listed in Table 2.

Accordingly a further aspect of the invention provides

7-[4-(4-chlorobenzyloxy)benzenesulfonyl]-8-methoxy-3-methyl-2,3,4,5-tetrahydro-1H-3-benzazepinium(1:1) maleate, dihydrate having an XRPD pattern with signals substantially as listed in Table 3.

Accordingly a further aspect of the invention provides

7-[4-(4-chlorobenzyloxy)benzenesulfonyl]-8-methoxy-3-methyl-2,3,4,5-tetrahydro-1H-3-benzazepinium(1:1) maleate, acetic acid solvate having an XRPD pattern with signals substantially as listed in Table 4.

The present invention also provides one or more chemical entities selected from the maleate and a pharmaceutically acceptable solvate thereof and the tosylate and a pharmaceutically acceptable solvate thereof when admixed with other material, for example another polymorphic form of the compound of formula (I).

The maleate salt of a compound of formula (I) may be prepared by contacting appropriate stoichiometric amounts of 7-[4-(4-chlorobenzyloxy)benzenesulfonyl]-8-methoxy-3-methyl-2,3,4,5-tetrahydro-1H-3-benzazepine free base with maleic acid in a suitable solvent. The tosylate salt of a compound of formula (I) may be prepared by contacting appropriate stoichiometric amounts of 7-[4-(4-chlorobenzyloxy)benzenesulfonyl]-8-methoxy-3-methyl-2,3,4,5-tetrahydro-1H-3-benzazepine free base with p-toluenesulfonic acid in a suitable solvent. The free base of 7-[4-(4-chlorobenzyloxy)benzenesulfonyl]-8-methoxy-3-methyl-2,3,4,5-tetrahydro-1H-3-benzazepine may for example be in solution with the appropriate acid added as a solid or both the free base of 7-[4-(4-chlorobenzyloxy)benzenesulfonyl]-8-methoxy-3-methyl-2,3,4,5-tetrahydro-1H-3-benzazepine and the appropriate acid may independently be in solution.

Suitable solvents for solubilising 7-[4-(4-chlorobenzyloxy)-benzenesulfonyl]-8-methoxy-3-methyl-2,3,4,5-tetrahydro-1H-3-benzazepine free base include for example alcohols such as ethanol, ketones such as acetone, halogenated hydrocarbons such as dichloromethane, and ethers such as tetrahydrofuran. If the maleic acid or the p-toluenesulfonic acid are to each be added as a solution in a solvent, the solvent used may include acetone, ethanol, methanol, propan-2-ol or water.

For the preparation of the maleate and the tosylate salt, the concentration of 7-[4-(4-chlorobenzyloxy)benzenesulfonyl]-8-methoxy-3-methyl-2,3,4,5-tetrahydro-1H-3-benzazepine free base may be for example in the range 3 to 25% weight/volume, such as in the range 5 to 15% weight/volume. The concentration of maleic acid or p-toluenesulfonic acid when used in solution may be for example in the range 0.5 to 5 molar. Elevated temperatures (for example up to the boiling point of the solvent used) may be used to increase the solubility of the free base and/or the acid.

The maleate salt and the tosylate salt may each be isolated in solid form by conventional means from a solution thereof obtained as above. For example, a non-crystalline salt may be prepared by precipitation from solution, spray drying or freeze drying of solutions, evaporating a solution to a glass, or vacuum drying of oils, or solidification of melts obtained from reaction of the free base and the acid.

Crystalline maleate salt and crystalline tosylate salt may each be prepared by directly crystallising from a solvent in which the salt has limited solubility, or by triturating or otherwise crystallising a non-crystalline salt. For example, 7-[4-(4-chlorobenzyloxy)benzenesulfonyl]-8-methoxy-3-methyl-2,3,4,5-tetrahydro-1H-3-benzazepinium maleate may be recrystallised from a variety of organic solvents, such as acetone, acetonitrile, butanone, 1-butanol, ethanol, 1-propanol or tetrahydrofuran or mixtures of such solvents. An improved yield of the salts may be obtained by the evaporation of some or all of the solvent or by crystallisation at elevated temperature followed by controlled cooling, for example in stages. Careful control of the precipitation temperature and seeding may be used to improve the reproducibility of the production process and the particle size distribution and form of the product. Individual polymorphs may be for example crystallized directly from a solution of the salt, although recrystallizing a solution of one polymorph using seeds of another polymorph may also be carried out.

In a further aspect of the invention there is provided a process for the preparation of the maleate salt of 7-[4-(4-chlorobenzyloxy)benzenesulfonyl]-8-methoxy-3-methyl-2,3,4,5-tetrahydro-1H-3-benzazepine comprising reacting 7-[4-(4-chlorobenzyloxy)benzene-sulfonyl]-8-methoxy-3-methyl-2,3,4,5-tetrahydro-1H-3-benzazepine free base with maleic acid in a suitable solvent, for example, ethanol.

In a still further aspect of the invention there is provided a process for the preparation of the tosylate salt of 7-[4-(4-chlorobenzyloxy)benzenesulfonyl]-8-methoxy-3-methyl-2,3,4,5-tetrahydro-1H-3-benzazepine comprising reacting 7-[4-(4-chlorobenzyloxy)benzene-sulfonyl]-8-methoxy-3-methyl-2,3,4,5-tetrahydro-1H-3-benzazepine free base with p-toluenesulfonic acid in a suitable solvent, for example, acetone.

7-[4-(4-Chlorobenzyloxy)benzenesulfonyl]-8-methoxy-3-methyl-2,3,4,5-tetrahydro-1H-3-benzazepine free base may be prepared by the following process as set forth in Scheme 1 or by processes disclosed in WO03/99786 which is incorporated herein by reference.

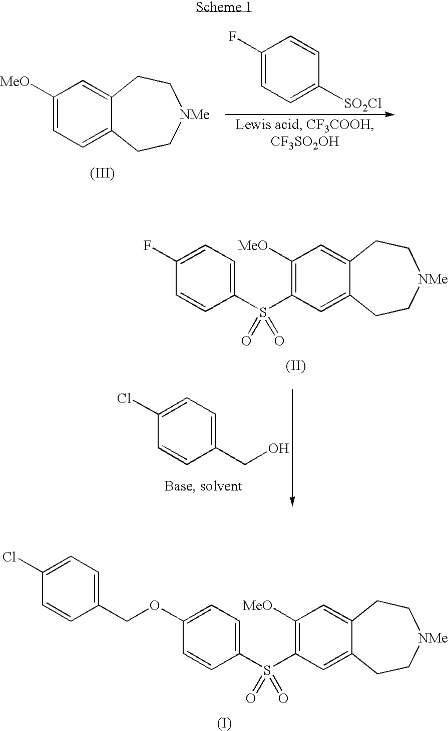

The compound of formula (I) may be prepared via the reaction of 4-chlorobenzyl alcohol in the presence of a base, for example sodium hydride or potassium tert-butoxide, with a compound of formula (II), in a suitable solvent, for example dimethyl sulfoxide or tetrahydrofuran.

Compounds of formula (II) may be prepared by reacting a compound of formula (III) with 4-fluorobenzenesulfonyl chloride in the presence of a Lewis acid, for example, indium(III) trifluromethanesulfonate, tin(II) trifluromethanesulfonate, bismuth(III) chloride, or indium(III) chloride, or mixtures thereof, and trifluoromethanesulfonic acid in a suitable solvent, for example, trifluoroacetic acid and, optionally, a co-solvent, for example dichloromethane.

A compound of formula (III) may be prepared using methods as described in the literature, for example using the route as described in European Patent EP285287. 4-Chlorobenzyl alcohol and 4-fluorobenzenesulfonyl chloride may be prepared according to known methods and/or are commercially available. Maleic acid and p-toluenesulfonic acid are commercially available.

Solvates of the maleate salt and the tosylate salt may each be prepared by conventional means from a solution of the maleate or tosylate salt. For example the dihydrate of the maleate salt may be prepared by recrystallisation of the maleate salt from a mixture of ethanol and water, for example in a ratio of 1:9. The acetic acid solvate of the maleate salt may be prepared by dissolving the maleate salt in a suitable quantity of acetic acid either at room temperature or elevated temperatures (for example up to the boiling point of the solvent used). Following dissolution of the salt, the resulting solution is allowed to stand at room temperature until crystallisation occurs.

As used herein, the phrase “the maleate salt and a pharmaceutically acceptable solvate thereof” is intended to include either the maleate salt, a pharmaceutically acceptable solvate of the maleate salt or mixtures of the maleate salt and one or more pharmaceutically acceptable solvates. Likewise, the phrase “the tosylate salt and a pharmaceutically acceptable solvate thereof” is intended to include either the tosylate salt, a pharmaceutically acceptable solvate of the tosylate salt or mixtures of the tosylate salt and one or more pharmaceutically acceptable solvates.

### DESCRIPTION OF FIGURES

FIG. 1 shows X-Ray powder diffraction (XRPD) data obtained for the maleate prepared as described in Example 1.

The maleate as described in Example 1 is characterised by having an XRPD pattern with signals substantially as listed in Table 1.

FIG. 2 shows the Raman spectrum of 7-[4-(4-chlorobenzyloxy)benzenesulfonyl]-8-methoxy-3-methyl-2,3,4,5-tetrahydro-1H-3-benzazepinium maleate prepared as described in Example 1.

FIG. 3 shows a Differential Scanning Calorimetry (DSC) thermogram of 7-[4-(4-Chlorobenzyloxy)benzenesulfonyl]-8-methoxy-3-methyl-2,3,4,5-tetrahydro-1H-3-benzazepinium maleate prepared as described in Example 1.

FIG. 4 shows XRPD data obtained for the tosylate prepared as described in Example 2.

The tosylate as described in Example 2 is characterised by having an XRPD pattern with signals substantially as listed in Table 2.

FIG. 5 shows the Raman spectrum of 7-[4-(4-chlorobenzyloxy)benzenesulfonyl]-8-methoxy-3-methyl-2,3,4,5-tetrahydro-1H-3-benzazepinium tosylate prepared as described in Example 2.

FIG. 6 shows a DSC thermogram of 7-[4(4-chlorobenzyloxy)benzenesulfonyl]-8-methoxy-3-methyl-2,3,4,5-tetrahydro-1H-3-benzazepinium tosylate prepared as described in Example 2.

FIG. 7 shows X-Ray powder diffraction (XRPD) data obtained for 7-[4-(4-chloro-benzyloxy)benzenesulfonyl]-8-methoxy-3-methyl-2,3,4,5-tetrahydro-1H-3-benzazepinium maleate, dihydrate prepared as described in Example 3.

The maleate, dihydrate as described in Example 3 is characterised by having an XRPD pattern with signals substantially as listed in Table 3.

FIG. 8 shows the Raman spectrum of 7-[4-(4-chlorobenzyloxy)benzenesulfonyl]-8-methoxy-3-methyl-2,3,4,5-tetrahydro-1H-3-benzazepinium maleate, dihydrate prepared as described in Example 3.

FIG. 9 shows a Differential Scanning Calorimetry (DSC) thermogram of 7-[4-(4-chloro-benzyloxy)benzenesulfonyl]-8-methoxy-3-methyl-2,3,4,5-tetrahydro-1H-3-benzazepinium maleate, dihydrate prepared as described in Example 3.

FIG. 10 shows X-Ray powder diffraction (XRPD) data obtained for 7-[4-(4-chloro-benzyloxy)benzenesulfonyl]-8-methoxy-3-methyl-2,3,4,5-tetrahydro-1H-3-benzazepinium maleate, acetic acid solvate prepared as described in Example 4.

The maleate, acetic acid solvate as described in Example 4 is characterised by having an XRPD pattern with signals substantially as listed in Table 4.

FIG. 11 shows the Raman spectrum of 7-[4-(4-chlorobenzyloxy)benzenesulfonyl]-8-methoxy-3-methyl-2,3,4,5-tetrahydro-1H-3-benzazepinium maleate, acetic acid solvate prepared as described in Example 4.

FIG. 12 shows a Differential Scanning Calorimetry (DSC) thermogram of 7-[4-(4-chloro-benzyloxy)benzenesulfonyl]-8-methoxy-3-methyl-2,3,4,5-tetrahydro-1H-3-benzazepinium maleate, acetic acid solvate prepared as described in Example 4.

It will be recognised that spectra and diffraction data will vary slightly according to various factors such as the temperature, concentration and instrumentation used. The skilled person will recognise that XRPD peak positions are affected by differences in sample height. The peak positions quoted herein are thus subject to a variation of ±0.15 degrees 2-theta.

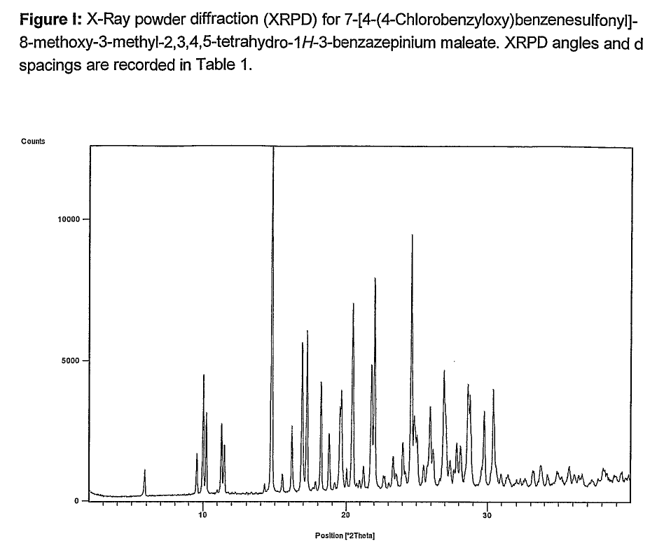

The present invention also provides the anhydrous maleate salt of 7-[4-(4-chlorobenzyloxy)-benzenesulfonyl]-8-methoxy-3-methyl-2,3,4,5-tetrahydro-1H-3-benzazepine characterised in that it provides an XRPD pattern substantially as illustrated in FIG. 1.

The present invention further provides the anhydrous maleate salt of 7-[4-(4-chlorobenzyloxy)benzenesulfonyl]-8-methoxy-3-methyl-2,3,4,5-tetrahydro-1H-3-benzazepine characterised in that it provides an XRPD pattern with signals substantially as listed in Table 1.

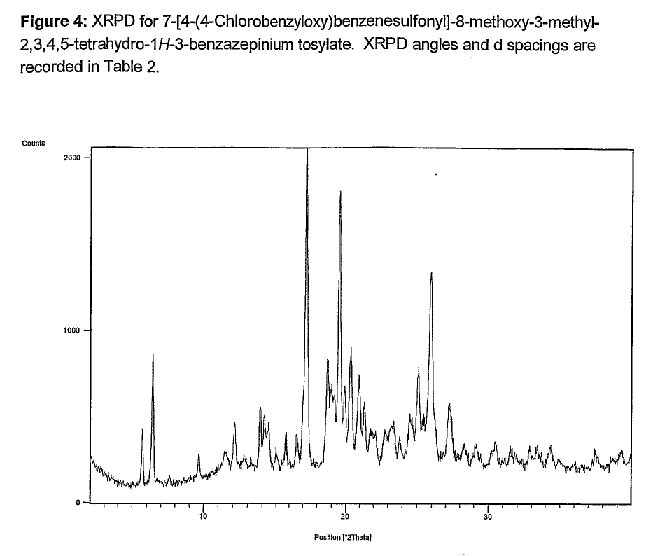

The present invention also provides the anhydrous tosylate salt of 7-[4-(4-chlorobenzyloxy)-benzenesulfonyl]-8-methoxy-3-methyl-2,3,4,5-tetrahydro-1H-3-benzazepine characterised in that it provides an XRPD pattern substantially as illustrated in FIG. 4.

The present invention further provides the anhydrous tosylate salt of 7-[4-(4-chlorobenzyloxy)benzenesulfonyl]-8-methoxy-3-methyl-2,3,4,5-tetrahydro-1H-3-benzazepine characterised in that it provides an XRPD pattern with signals substantially as listed in Table 2.

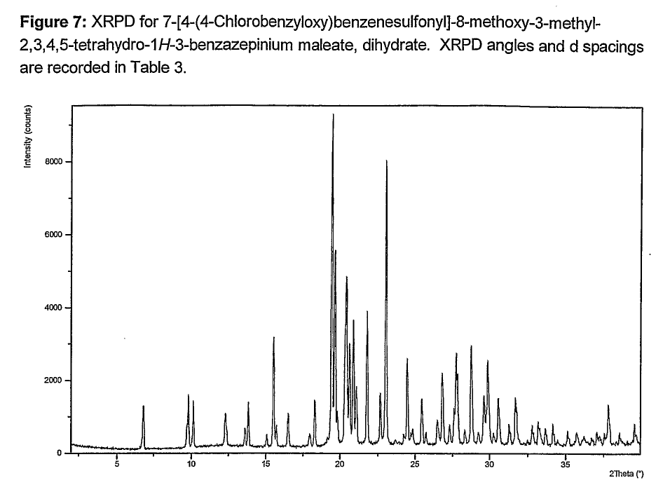

The present invention also provides the dihydrate of the maleate salt of 7-[4-(4-chlorobenzyloxy)-benzenesulfonyl]-8-methoxy-3-methyl-2,3,4,5-tetrahydro-1H-3-benzazepine characterised in that it provides an XRPD pattern substantially as illustrated in FIG. 7.

The present invention further provides the dihydrate of the maleate salt of 7-[4-(4-chlorobenzyloxy)benzenesulfonyl]-8-methoxy-3-methyl-2,3,4,5-tetrahydro-1H-3-benzazepine characterised in that it provides an XRPD pattern with signals substantially as listed in Table 3.

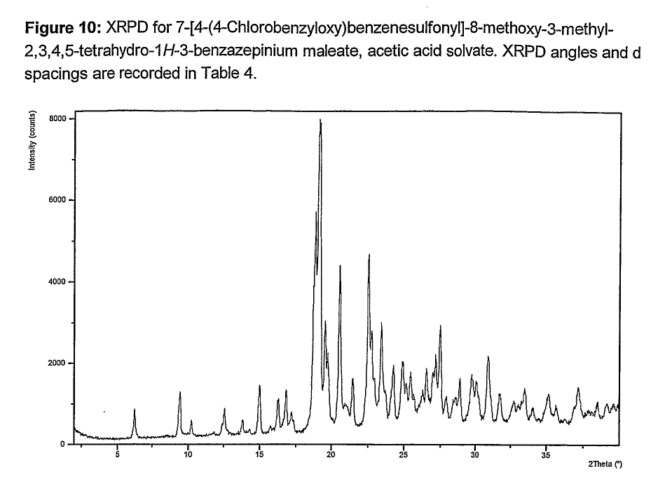

The present invention also provides the acetic acid solvate of the maleate salt of 7-[4-(4-chlorobenzyloxy)-benzenesulfonyl]-8-methoxy-3-methyl-2,3,4,5-tetrahydro-1H-3-benzazepine characterised in that it provides an XRPD pattern substantially as illustrated in FIG. 10.

The present invention further provides the acetic acid solvate of the maleate salt of 7-[4-(4-chlorobenzyloxy)benzenesulfonyl]-8-methoxy-3-methyl-2,3,4,5-tetrahydro-1H-3-benzazepine characterised in that it provides an XRPD pattern with signals substantially as listed in Table 4.

7-[4-(4-Chlorobenzyloxy)benzenesulfonyl]-8-methoxy-3-methyl-2,3,4,5-tetrahydro-1H-3-benzazepinium maleate and tosylate salts and pharmaceutically acceptable solvates thereof have been found to exhibit affinity for dopamine receptors, in particular the D₃ and D₂ receptors, and are useful in the treatment of disease states which require modulation of such receptors, such as psychotic conditions. These salts have also been found to have greater affinity for dopamine D₃ than for D₂ receptors. The therapeutic effect of currently available antipsychotic agents (neuroleptics) is generally believed to be exerted via blockade of D₂ receptors; however this mechanism is also thought to be responsible for undesirable extrapyramidal side effects (eps) associated with many neuroleptic agents. Without wishing to be bound by theory, it has been suggested that blockade of the dopamine D₃ receptor may give rise to beneficial antipsychotic activity without significant eps (see for example Sokoloff et al, Nature, 1990; 347: 146-151; and Schwartz et al, Clinical Neuropharmacology, Vol 16, No. 4, 295-314, 1993).

7-[4-(4-Chlorobenzyloxy)benzenesulfonyl]-8-methoxy-3-methyl-2,3,4,5-tetrahydro-1H-3-benzazepinium maleate and tosylate salts and pharmaceutically acceptable solvates thereof have also been found to have antagonist affinity for the serotonin 5-HT_{2C}, 5-HT_{2A} and 5-HT₆ receptors. These properties may give rise to anti-psychotic activity (e.g. improved effects on cognitive dysfunction) activity with reduced eps, and/or anxiolytic/antidepressant activity. These could include, but are not limited to, attenuation of cognitive symptoms via 5-HT₆ receptor blockade (see Reavill, C. and Rogers, D.C., 2001, Investigational Drugs 2, 104-109), and reduced anxiety (see for example Kennett et al., Neuropharmacology 1997 April-May; 36 (4-5): 609-20), protection against eps (Reavill et al., Brit. J. Pharmacol., 1999; 126: 572-574) and antidepressant activity (Bristow et al., Neuropharmacology 39:2000; 1222-1236) via 5-HT_{2C} receptor blockade.

7-[4-(4-Chlorobenzyloxy)benzenesulfonyl]-8-methoxy-3-methyl-2,3,4,5-tetrahydro-1H-3-benzazepinium maleate and tosylate salts and pharmaceutically acceptable solvates thereof may also exhibit affinity for other receptors not mentioned above, resulting in beneficial antipyschotic activity.

The maleate or tosylate salts of 7-[4-(4-Chlorobenzyloxy)benzenesulfonyl]-8-methoxy-3-methyl-2,3,4,5-tetrahydro-1H-3-benzazepine or their pharmaceutically acceptable solvates thereof are of use in the treatment of psychotic disorders.

In a further aspect therefore, the invention provides one or more chemical entities selected from the maleate and tosylate salt of 7-[4-(4-Chlorobenzyloxy)benzenesulfonyl]-8-methoxy-3-methyl-2,3,4,5-tetrahydro-1H-3-benzazepine and pharmaceutically acceptable solvates thereof for use in therapy.

In another aspect, the invention provides one or more chemical entities selected from the maleate and tosylate salt of 7-[4-(4-Chlorobenzyloxy)benzenesulfonyl]-8-methoxy-3-methyl-2,3,4,5-tetrahydro-1H-3-benzazepine and pharmaceutically acceptable solvates thereof for use in the treatment of a condition which requires modulation of a dopamine receptor.

In another aspect, the invention provides one or more chemical entities selected from the maleate and tosylate salts of 7-[4-(4-Chlorobenzyloxy)benzenesulfonyl]-8-methoxy-3-methyl-2,3,4,5-tetrahydro-1H-3-benzazepine and pharmaceutically acceptable solvates thereof for use in the treatment of psychotic disorders.

In another aspect, the invention provides the use of one or more chemical entities selected from the maleate and tosylate salt of 7-[4-(4-Chlorobenzyloxy)benzenesulfonyl]-8-methoxy-3-methyl-2,3,4,5-tetrahydro-1H-3-benzazepine and pharmaceutically acceptable solvates thereof in the manufacture of a medicament for the treatment of a condition which requires modulation of a dopamine receptor.

In another aspect, the invention provides the use of one or more chemical entities selected from the maleate and tosylate salt of 7-[4-(4-Chlorobenzyloxy)benzenesulfonyl]-8-methoxy-3-methyl-2,3,4,5-tetrahydro-1H-3-benzazepine and pharmaceutically acceptable solvates thereof in the manufacture of a medicament for the treatment of psychotic disorders.

In another aspect, the invention provides a method of treating a condition which requires modulation of a dopamine receptor, which comprises administering to a mammal in need thereof an effective amount of one or more chemical entities selected from the maleate and tosylate salt of 7-[4-(4-Chlorobenzyloxy)benzenesulfonyl]-8-methoxy-3-methyl-2,3,4,5-tetrahydro-1H-3-benzazepine and pharmaceutically acceptable solvates thereof.

In another aspect, the invention provides a method of treating psychotic disorders which comprises administering to a mammal in need thereof an effective amount of one or more chemical entities selected from the maleate and tosylate salt of 7-[4-(4-Chlorobenzyloxy)benzenesulfonyl]-8-methoxy-3-methyl-2,3,4,5-tetrahydro-1H-3-benzazepine and pharmaceutically acceptable solvates thereof.

Within the context of the present invention, the terms describing the indications used herein are classified in the Diagnostic and Statistical Manual of Mental Disorders, 4th Edition, published by the American Psychiatric Association (DSM-IV) and/or the International Classification of Diseases, 10th Edition (ICD-10). The various subtypes of the disorders mentioned herein are contemplated as part of the present invention. Numbers in brackets after the listed diseases below refer to the classification code in DSM-IV.

Within the context of the present invention, the term “psychotic disorder” includes:

Schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-induced Psychotic Disorder including the subtypes With Delusions (293.81) and With Hallucinations (293.82); and Psychotic Disorder Not Otherwise Specified (298.9).

The maleate and tosylate salts of 7-[4-(4-Chlorobenzyloxy)benzenesulfonyl]-8-methoxy-3-methyl-2,3,4,5-tetrahydro-1H-3-benzazepine and pharmaceutically acceptable solvates thereof may also be of use in the treatment of the following disorders:

Depression and mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-induced Mood Disorder (including the subtypes With Depressive Features, With Manic Features and With Mixed Features) and Mood Disorder Not Otherwise Specified (296.90):

Anxiety disorders including Social Anxiety Disorder, Panic Attack, Agoraphobia, Panic Disorder, Agoraphobia Without History of Panic Disorder (300.22), Specific Phobia (300.29) including the subtypes Animal Type, Natural Environment Type, Blood-Injection-Injury Type, Situational Type and Other Type), Social Phobia (300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder and Anxiety Disorder Not Otherwise Specified (300.00):

Substance-related disorders including Substance Use Disorders such as Substance Dependence, Substance Craving and Substance Abuse; Substance-induced Disorders such as Substance Intoxication, Substance Withdrawal, Substance-induced Delirium, Substance-Induced Persisting Dementia, Substance-induced Persisting Amnestic Disorder, Substance-Induced Psychotic Disorder, Substance-induced Mood Disorder, Substance-induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-induced Persisting Dementia, Alcohol-Induced Persisting Amnestic Disorder, Alcohol-Induced Psychotic Disorder, Alcohol-induced Mood Disorder, Alcohol-Induced Anxiety Disorder, Alcohol-induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-induced Psychotic Disorder, Hallucinogen-induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic- Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide:

Sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition; and Substance-Induced Sleep Disorder including the subtypes Insomnia Type, Hypersomnia Type, Parasomnia Type and Mixed Type:

Eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; and Eating Disorder Not Otherwise Specified (307.50):

Autistic Disorder (299.00); Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23):

Personality Disorders including the subtypes Paranoid Personality Disorder (301.0), Schizoid Personality Disorder (301.20), Schizotypal Personality Disorder (301,22), Antisocial Personality Disorder (301.7), Borderline Personality Disorder (301,83), Histrionic Personality Disorder (301.50), Narcissistic Personality Disorder (301,81), Avoidant Personality Disorder (301.82), Dependent Personality Disorder (301.6), Obsessive-Compulsive Personality Disorder (301.4) and Personality Disorder Not Otherwise Specified (301.9):

Enhancement of cognition including the treatment of cognition impairment in other diseases such as schizophrenia, bipolar disorder, depression, other psychiatric disorders and psychotic conditions associated with cognitive impairment, e.g. Alzheimer's disease: and

Sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302-85); and Sexual Disorder Not Otherwise Specified (302.9).

All of the various forms and sub-forms of the disorders mentioned herein are contemplated as part of the present invention.

“Treatment” includes prophylaxis, where this is appropriate for the relevant condition(s).

It will be appreciated by those skilled in the art that one or more chemical entities selected from maleate and tosylate salts of 7-[4-(4-Chlorobenzyloxy)benzenesulfonyl]-8-methoxy-3-methyl-2,3,4,5-tetrahydro-1H-3-benzazepine and their pharmaceutically acceptable solvates thereof according to the invention may advantageously be used in conjunction with one or more other therapeutic agents, for instance, 5HT₃ antagonists, serotonin agonists, NK-1 antagonists, selective serotonin reuptake inhibitors (SSRI), noradrenaline re-uptake inhibitors (SNRI), non-selective reuptake inhibitors of one or more of serotonin, noradrenaline and norepinephrine, CRF-1 antagonists, tricyclic antidepressants, dopaminergic antidepressants, H₃ antagonists, 5HT_{1A} antagonists, 5HT_{1B} antagonists, 5HT_{1D} antagonists, 5HT₄ partial agonists, D1 agonists, M1 agonists, anticonvulsant agents and/or cyclooxygenase-2 (COX-2) inhibitors.

It will be appreciated that the compounds of the combination or composition may be administered simultaneously (either in the same or different pharmaceutical formulations), separately or sequentially.

Suitable 5HT₃ antagonists which may be used in combination with the maleate or tosylate salt of 7-[4-(4-Chlorobenzyloxy)benzenesulfonyl]-8-methoxy-3-methyl-2,3,4,5-tetrahydro-1H-3-benzazepine and pharmaceutically acceptable solvates thereof include for example one or more chemical entities selected from ondansetron, granisetron and metoclopramide.

Suitable serotonin agonists which may be used in combination with the maleate or tosylate salt of 7-[4-(4-Chlorobenzyloxy)benzenesulfonyl]-8-methoxy-3-methyl-2,3,4,5-tetrahydro-1H-3-benzazepine and pharmaceutically acceptable solvates thereof include for example one or more chemical entities selected from sumatriptan, rauwolscine, yohimbine and metoclopramide.

Suitable SSRIs which may be used in combination with the maleate or tosylate salt of 7-[4-(4-Chlorobenzyloxy)benzenesulfonyl]-8-methoxy-3-methyl-2,3,4,5-tetrahydro-1H-3-benzazepine and pharmaceutically acceptable solvates thereof include for example one or more chemical entities selected from fluoxetine, citalopram, femoxetine, fluvoxamine, paroxetine, indalpine, sertraline and zimeldine.

Suitable SNRIs which may be used in combination with the maleate or tosylate salt of 7-[4-(4-Chlorobenzyloxy)benzenesulfonyl]-8-methoxy-3-methyl-2,3,4,5-tetrahydro-1H-3-benzazepine and pharmaceutically acceptable solvates thereof include for example one or more chemical entities selected from venlafaxine and reboxetine.

Suitable tricyclic antidepressants which may be used in combination with the maleate or tosylate salt of 7-[4-(4-Chlorobenzyloxy)benzenesulfonyl]-8-methoxy-3-methyl-2,3,4,5-tetrahydro-1H-3-benzazepine and pharmaceutically acceptable solvates thereof include for example one or more chemical entities selected from imipramine, amitriptiline, chlomipramine and nortriptiline.

Suitable dopaminergic antidepressants which may be used in combination with the maleate or tosylate salt of 7-[4-(4-Chlorobenzyloxy)benzenesulfonyl]-8-methoxy-3-methyl-2,3,4,5-tetrahydro-1H-3-benzazepine and pharmaceutically acceptable solvates thereof include for example one or more chemical entities selected from bupropion and amineptine.

Suitable anticonvulsant agents which may be used in combination with the maleate or tosylate salt of 7-[4-(4-Chlorobenzyloxy)benzenesulfonyl]-8-methoxy-3-methyl-2,3,4,5-tetrahydro-1H-3-benzazepine and pharmaceutically acceptable solvates thereof include for example one or more chemical entities selected from divalproex, carbamazepine and diazepam.

Suitable NSAID agents which may be used in combination with the maleate or tosylate salt of 7-[4-(4-Chlorobenzyloxy)benzenesulfonyl]-8-methoxy-3-methyl-2,3,4,5-tetrahydro-1H-3-benzazepine and pharmaceutically acceptable solvates thereof include for example one or more chemical entities selected from ibuprofen, aspirin and its active metabolite salicylate.

Suitable COX-2 inhibitors which may be used in combination of the maleate or tosylate salt of 7-[4-(4-Chlorobenzyloxy)benzenesulfonyl]-8-methoxy-3-methyl-2,3,4,5-tetrahydro-1H-3-benzazepine and pharmaceutically acceptable solvates thereof include for example rofecoxib (available under the tradename VIOXX®, from Merck, U.S. Pat. No. 5,474,995); celecoxib (available under the tradename CELEBREX®, from Pfizer, U.S. Pat. No. 5,466,823); valdecoxib (available under the tradename BEXTRA®, from Pfizer, U.S. Pat. No. 6,633,272); etoricoxib (available under the tradename ARCOXIA®, from Merck, U.S. Pat. No. 5,861,419); lumiracoxib (available under the tradename PREXIGE®, from Novartis); paracoxib (U.S. Pat. No. 5,932,598); COX-189 from Novartis; BMS347070 from Bristol Myers Squibb; tiracoxib (JTE522) from Japan Tobacco; ABT963 from Abbott; CS502 from Sankyo; 2-(4-ethoxyphenyl)-3-(3-methanesulfonylphenyl)-pyrazolo[1,5-b]pyridazine (GlaxoSmithKline) and 2-butoxy-4-[4-(methylsulfonyl)phenyl]-6-(trifluoromethyl)pyrimidine (GlaxoSmithKiine).

The maleate or tosylate salts of 7-[4-(4-Chlorobenzyloxy)benzenesulfonyl]-8-methoxy-3-methyl-2,3,4,5-tetrahydro-1H-3-benzazepine and their pharmaceutically acceptable solvates are also suitable for combination with other typical and atypical antipsychotics to provide improved treatment of psychotic disorders. Particular advantages associated with the combinations, uses and methods of treatment of the maleate or tosylate salts of 7-[4-(4-Chlorobenzyloxy)benzenesulfonyl]-8-methoxy-3-methyl-2,3,4,5-tetrahydro-1H-3-benzazepine or their pharmaceutically acceptable solvates include equivalent or improved efficacy at doses of administration which are lower than those commonly used for the individual components. Improved treatments of positive symptoms and/or negative symptoms and/or cognitive symptoms of the psychotic disorder may also be observed. The combinations, uses and methods of treatment of the invention may also provide advantages in treatment of patients who fail to respond adequately or who are resistant to treatment with certain antipsychotic agents (also known as neuroleptic agents).

The combination therapies of the invention are preferably administered adjunctively. By adjunctive administration is meant the coterminous or overlapping administration of each of the components in the form of separate pharmaceutical compositions or devices. This regime of therapeutic administration of two or more therapeutic agents is referred to generally by those skilled in the art and herein as adjunctive therapeutic administration; it is also known as add-on therapeutic administration. Any and all treatment regimes in which a patient receives separate but coterminous or overlapping therapeutic administration of the maleate or tosylate salts of 7-[4-(4-Chlorobenzyloxy)benzenesulfonyl]-8-methoxy-3-methyl-2,3,4,5-tetrahydro-1H-3-benzazepine or their pharmaceutically acceptable solvates and at least one antipsychotic agent are within the scope of the current invention. In one embodiment of adjunctive therapeutic administration as described herein, a patient is typically stabilised on a therapeutic administration of one or more of the components for a period of time and then receives administration of another component. The maleate or tosylate salts of 7-[4-(4-Chlorobenzyloxy)benzenesulfonyl]-8-methoxy-3-methyl-2,3,4,5-tetrahydro-1H-3-benzazepine or their pharmaceutically acceptable solvates may be administered as adjunctive therapeutic treatment to patients who are receiving administration of at least one antipsychotic agent, but the scope of the invention also includes the adjunctive therapeutic administration of at least one antipsychotic agent to patients who are receiving administration of the maleate or tosylate salt of the compound of formula (I) or pharmaceutically acceptable solvates thereof.

The combination therapies of the invention may also be administered simultaneously. By simultaneous administration is meant a treatment regime wherein the individual components are administered together, either in the form of a single pharmaceutical composition or device comprising or containing both components, or as separate compositions or devices, each comprising one of the components, administered simultaneously. Such combinations of the separate individual components for simultaneous combination may be provided in the form of a kit-of-parts.

In a further aspect therefore, the invention provides a method of treatment of a psychotic disorder by adjunctive therapeutic administration of the maleate or tosylate salts of 7-[4-(4-Chlorobenzyloxy)benzenesulfonyl]-8-methoxy-3-methyl-2,3,4,5-tetrahydro-1H-3-benzazepine or their pharmaceutically acceptable solvates to a patient receiving therapeutic administration of at least one antipsychotic agent. In a further aspect, the invention provides the use of the maleate or tosylate salts of 7-[4-(4-Chlorobenzyloxy)benzenesulfonyl]-8-methoxy-3-methyl-2,3,4,5-tetrahydro-1H-3-benzazepine or their pharmaceutically acceptable solvates in the manufacture of a medicament for adjunctive therapeutic administration for the treatment of a psychotic disorder in a patient receiving therapeutic administration of at least one antipsychotic agent. The invention further provides the maleate or tosylate salts of 7-[4-(4-Chlorobenzyloxy)benzenesulfonyl]-8-methoxy-3-methyl-2,3,4,5-tetrahydro-1H-3-benzazepine or their pharmaceutically acceptable solvates for use for adjunctive therapeutic administration for the treatment of a psychotic disorder in a patient receiving therapeutic administration of at least one antipsychotic agent.

In a further aspect, the invention provides a method of treatment of a psychotic disorder by adjunctive therapeutic administration of at least one antipsychotic agent to a patient receiving therapeutic administration of the maleate or tosylate salts of 7-[4-(4-Chlorobenzyloxy)benzenesulfonyl]-8-methoxy-3-methyl-2,3,4,5-tetrahydro-1H-3-benzazepine or their pharmaceutically acceptable solvates. In a further aspect, the invention provides the use of at least one antipsychotic agent in the manufacture of a medicament for adjunctive therapeutic administration for the treatment of a psychotic disorder in a patient receiving therapeutic administration of the maleate or tosylate salts of 7-[4-(4-Chlorobenzyloxy)benzenesulfonyl]-8-methoxy-3-methyl-2,3,4,5-tetrahydro-1H-3-benzazepine or their pharmaceutically acceptable solvates. The invention further provides at least one antipsychotic agent for adjunctive therapeutic administration for the treatment of a psychotic disorder in a patient receiving therapeutic administration of the maleate or tosylate salts of 7-[4-(4-Chlorobenzyloxy)benzenesulfonyl]-8-methoxy-3-methyl-2,3,4,5-tetrahydro-1H-3-benzazepine or their pharmaceutically acceptable solvates.

In a further aspect, the invention provides a method of treatment of a psychotic disorder by simultaneous therapeutic administration of the maleate or tosylate salts of 7-[4-(4-Chlorobenzyloxy)benzenesulfonyl]-8-methoxy-3-methyl-2,3,4,5-tetrahydro-1H-3-benzazepine or their pharmaceutically acceptable solvates in combination with at least one antipsychotic agent. The invention further provides the use of a combination of the maleate or tosylate salts of 7-[4-(4-Chlorobenzyloxy) benzenesulfonyl]-8-methoxy-3-methyl-2,3,4,5-tetrahydro-1H-3-benzazepine or their pharmaceutically acceptable solvates and at least one antipsychotic agent in the manufacture of a medicament for simultaneous therapeutic administration in the treatment of a psychotic disorder. The invention further provides the use of the maleate or tosylate salts of 7-[4-(4-Chlorobenzyloxy)benzenesulfonyl]-8-methoxy-3-methyl-2,3,4,5-tetrahydro-1H-3-benzazepine or their pharmaceutically acceptable solvates in the manufacture of a medicament for simultaneous therapeutic administration with at least one antipsychotic agent in the treatment of a psychotic disorder. The invention further provides the maleate or tosylate salts of 7-[4-(4-Chlorobenzyloxy)benzenesulfonyl]-8-methoxy-3-methyl-2,3,4,5-tetrahydro-1H-3-benzazepine or their pharmaceutically acceptable solvates for use for simultaneous therapeutic administration with at least one antipsychotic agent in the treatment of a psychotic disorder. The invention further provides the use of at least one antipsychotic agent in the manufacture of a medicament for simultaneous therapeutic administration with the maleate or tosylate salts of 7-[4-(4-Chlorobenzyloxy)benzenesulfonyl]-8-methoxy-3-methyl-2,3,4,5-tetrahydro-1H-3-benzazepine or their pharmaceutically acceptable solvates in the treatment of a psychotic disorder.

In further aspects, the invention provides a method of treatment of a psychotic disorder by simultaneous therapeutic administration of a pharmaceutical composition comprising the maleate or tosylate salts of 7-[4-(4-Chlorobenzyloxy)benzenesulfonyl]-8-methoxy-3-methyl-2,3,4,5-tetrahydro-1H-3-benzazepine or their pharmaceutically acceptable solvates and at least one mood stabilising or antimanic agent, a pharmaceutical composition comprising the maleate or tosylate salts of 7-[4-(4-Chlorobenzyloxy)benzenesulfonyl]-8-methoxy-3-methyl-2,3,4,5-tetrahydro-1H-3-benzazepine or their pharmaceutically acceptable solvates and at least one mood stabilising or antimanic agent, the use of a pharmaceutical composition comprising the maleate or tosylate salts of 7-[4-(4-Chlorobenzyloxy)benzenesulfonyl]-8-methoxy-3-methyl-2,3,4,5-tetrahydro-1H-3-benzazepine or their pharmaceutically acceptable solvates and at least one mood stabilising or antimanic agent in the manufacture of a medicament for the treatment of a psychotic disorder, and a pharmaceutical composition comprising the maleate or tosylate salts of 7-[4-(4-Chlorobenzyloxy)benzenesulfonyl]-8-methoxy-3-methyl-2,3,4,5-tetrahydro-1H-3-benzazepine or their pharmaceutically acceptable solvates and at least one mood stabilising or antimanic agent for use in the treatment of a psychotic disorder.

In a further aspect, the invention provides a kit-of-parts for use in the treatment of a psychotic disorder comprising a first dosage form comprising the maleate or tosylate salts of 7-[4-(4-Chlorobenzyloxy)benzenesulfonyl]-8-methoxy-3-methyl-2,3,4,5-tetrahydro-1H-3-benzazepine or their pharmaceutically acceptable solvates and one or more further dosage forms each comprising an antipsychotic agent for simultaneous therapeutic administration.

Examples of antipsychotic drugs that are useful in the present invention include, but are not limited to: butyrophenones, such as haloperidol, pimozide, and droperidol; phenothiazines, such as chlorpromazine, thioridazine, mesoridazine, trifluoperazine, perphenazine, fluphenazine, thiflupromazine, prochlorperazine, and acetophenazine; thioxanthenes, such as thiothixene and chlorprothixene; thienobenzodiazepines; dibenzodiazepines; benzisoxazoles; dibenzothiazepines; imidazolidinones; benziso-thiazolyl-piperazines; triazine such as lamotrigine; dibenzoxazepines, such as loxapine; dihydroindolones, such as molindone; aripiprazole; and derivatives thereof that have antipsychotic activity.

Examples of tradenames and suppliers of selected antipsychotic drugs that are suitable for use in the present invention are as follows: clozapine (available under the tradename CLOZARIL®, from Mylan, Zenith Goldline, UDL, Novartis); olanzapine (available under the tradename ZYPREXA®, from Lilly; ziprasidone (available under the tradename GEODON®, from Pfizer); risperidone (available under the tradename RISPERDAL®, from Janssen); quetiapine fumarate (available under the tradename SEROQUEL®, from AstraZeneca); sertindole (available under the tradename SERLECT®); amisuipride (available under the tradename SOLION®, from Sanofi-Synthelabo); haloperidol (available under the tradename HALDOL®, from Ortho-McNeil); haloperidol decanoate (available under the tradename HALDOL decanoate®); haloperidol lactate (available under the tradenames HALDOL® and INTENSOL®) chlorpromazine (available under the tradename THORAZINE®, from SmithKline Beecham (GSK); fluphenazine (available under the tradename PROLIXIN®, from Apothecon, Copley, Schering, Teva, and American Pharmaceutical Partners, Pasadena); fluphenazine decanoate (available under the tradename PROLIXIN decanoate®); fluphenazine enanthate (available under the tradename PROLIXIN®); fluphenazine hydrochloride (available under the tradename PROLIXIN®); thiothixene (available under the tradename NAVANE®; from Pfizer); thiothixene hydrochloride (available under the tradename NAVANE®); trifluoperazine (10-[3-(4-methyl-1-piperazinyl)propyl]-2-(trifluoromethyl)phenothiazine dihydrochloride, available under the tradename STELAZINE®, from SmithKlien Beckman; perphenazine (available under the tradename TRILAFON®; from Schering); perphenazine and amitriptyline hydrochloride (available under the tradename ETRAFON TRILAFON®); thioridazine (available under the tradename MELLARIL®; from Novartis, Roxane, HiTech, Teva, and Alpharma); molindone (available under the tradename MOBAN®, from Endo); molindone hydrochloride (available under the tradename MOBAN®); loxapine (available under the tradename LOXITANE®; from Watson); loxapine hydrochloride (available under the tradename LOXITANE®); and loxapine succinate (available under the tradename LOXITANE®). Furthermore, benperidol (Glianimon®), perazine (Taxilan®) or melperone (Eunerpan®)) may be used.

Other suitable antipsychotic drugs include promazine (available under the tradename SPARINE®), triflurpromazine (available under the tradename VESPRIN®), chlorprothixene (available under the tradename TARACTAN®), droperidol (available under the tradename INAPSINE®), acetophenazine (available under the tradename TINDAL®;), prochlorperazine (available under the tradename COMPAZINE®), methotrimeprazine (available under the tradename NOZINAN®), pipotiazine (available under the tradename PIPOTRIL®), iloperidone, pimozide and flupenthixol.

In one further aspect of the invention, suitable antipsychotic agents include olanzapine, risperidone, quetiapine, aripiprazole, haloperidol, clozapine, ziprasidone and osanetant.

For use in medicine, 7-[4-(4-chlorobenzyloxy)benzenesulfonyl]-8-methoxy-3-methyl-2,3,4,5-tetrahydro-1H-3-benzazepinium maleate or tosylate or a pharmaceutically acceptable solvate thereof are usually administered as a standard pharmaceutical composition. The pharmaceutical composition can be for use in the treatment of any of the conditions described herein.

Therefore in a further aspect of the present invention there is provided a pharmaceutical composition comprising one or more chemical entities selected from 7-[4-(4-chlorobenzyloxy)benzenesulfonyl]-8-methoxy-3-methyl-2,3,4,5-tetrahydro-1H-3-benzazepinium maleate and a pharmaceutically acceptable solvate thereof, together with a pharmaceutically acceptable carrier.

In another aspect of the present invention there is provided a pharmaceutical composition comprising one or more chemical entities selected from 7-[4-(4-chlorobenzyloxy)-benzenesulfonyl]-8-methoxy-3-methyl-2,3,4,5-tetrahydro-1H-3-benzazepinium tosylate and a pharmaceutically acceptable solvate thereof, together with a pharmaceutically acceptable carrier.

7-[4-(4-Chlorobenzyloxy)benzenesulfonyl]-8-methoxy-3-methyl-2,3,4,5-tetrahydro-1H-3-benzazepinium maleate or tosylate or a pharmaceutically acceptable solvate thereof may be administered by any convenient method, for example by oral, parenteral (e.g. intravenous), buccal, sublingual, nasal, rectal or transdermal administration and the pharmaceutical compositions adapted accordingly.

7-[4-(4-Chlorobenzyloxy)benzenesulfonyl]-8-methoxy-3-methyl-2,3,4,5-tetrahydro-1H-3-benzazepinium maleate or tosylate or a pharmaceutically acceptable solvate thereof can be formulated as liquids or solids, for example syrups, suspensions or emulsions, tablets, capsules and lozenges.

A liquid formulation will generally consist of a suspension or solution of 7-[4-(4-chlorobenzyloxy)benzenesulfonyl]-8-methoxy-3-methyl-2,3,4,5-tetrahydro-1H-3-benzazepinium maleate or tosylate or a pharmaceutically acceptable solvate thereof in a suitable liquid carrier(s) for example an aqueous solvent such as water, ethanol or glycerine, or a non-aqueous solvent, such as polyethylene glycol or an oil. The formulation may also contain a suspending agent, preservative, flavouring or colouring agent.

A composition in the form of a tablet can be prepared using any suitable pharmaceutical carrier(s) routinely used for preparing solid formulations. Examples of such carriers include magnesium stearate, starch, lactose, sucrose and cellulose.

A composition in the form of a capsule can be prepared using routine encapsulation procedures. For example, pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule.

Typical parenteral compositions consist of a solution or suspension of 7-[4-(4-chlorobenzyloxy)benzenesulfonyl]-8-methoxy-3-methyl-2,3,4,5-tetrahydro-1H-3-benzazepinium maleate or tosylate or a pharmaceutically acceptable solvate thereof in a sterile aqueous carrier or parenterally acceptable oil, for example polyethylene glycol, polyvinyl pyrrolidone, lecithin, arachis oil or sesame oil, Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the active substance in a pharmaceutically acceptable aqueous or non-aqueous solvent and are usually presented in single or multidose quantities in sterile form in a sealed container, which can take the form of a cartridge or refill for use with an atomising device. Alternatively the sealed container may be a unitary dispensing device such as a single dose nasal inhaler or an aerosol dispenser fitted with a metering valve which is intended for disposal once the contents of the container have been exhausted. Where the dosage form comprises an aerosol dispenser, it will contain a propellant which can be a compressed gas such as compressed air or an organic propellant such as a fluorochloro-hydrocarbon. The aerosol dosage forms can also take the form of a pump-atomiser.

Compositions suitable for buccal or sublingual administration include tablets, lozenges and pastilles, wherein the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches. The composition is suitably in unit dose form such as a tablet, capsule or ampoule.

7-[4-(4-Chlorobenzyloxy)benzenesulfonyl]-8-methoxy-3-methyl-2,3,4,5-tetrahydro-1H-3-benzazepinium maleate or tosylate or a pharmaceutically acceptable solvate thereof will normally be administered in a daily dosage regimen (for an adult patient) of, for example, an oral dose of between 1 mg and 250 mg, such as between 1 mg and 250 mg, such as between 2 mg and 100mg, e.g. between 2 and 50 mg or an intravenous, subcutaneous, or intramuscular dose of between 0.1 mg and 100 mg, for example between 0.1 mg and 50 mg, e.g. between 1 and 25 mg of 7-[4-(4-chlorobenzyloxy)benzenesulfonyl]-8-methoxy-3-methyl-2,3,4,5-tetrahydro-1H-3-benzazepinium calculated as the free base, the compound being administered 1 to 4 times per day. Suitably the compounds will be administered for a period of continuous therapy, for example for a week or more.

No adverse toxicological effects have been observed for compounds of the invention at doses expected to be approved for therapeutic administration.

The invention is further illustrated by the following non-limiting examples:

### Preparation of 7-[4-(4-Chlorobenzyloxy)benzenesulfonyl]-8-methoxy-3-methyl-2,3,4,5-tetrahydro-1H-3-benzazepine

### Description 1

### 8-Methoxy-3-methyl-2,3,4,5-tetrahydro-1H-3-benzazepine-7-sulfonyl fluoride (D1)

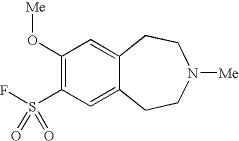

### a) 8-Methoxy-3-methyl-2,3,4,5-tetrahydro-1H-3-benzazepine-7-sulfonic acid

7-Methoxy-3-methyl-2,3,4,5-tetrahydro-1H-3-benzazepine (see EP 285287) (23 g) was dissolved in trifluoroacetic acid (125 mL), and then stirred in an ice bath while chlorosulfonic acid (16.5 mL, 250 mmol) was added dropwise. The solution was stirred for 30 minutes, then evaporated to dryness to afford the title sulfonic acid which was used directly in the next step.

### b) 8-Methoxy-3-methyl-2,3,4,5-tetrahydro-1H-3-benzazepine-7-sulfonyl chloride

The sulfonic acid from part (a) was dissolved in thionyl chloride (75 mL) and the solution refluxed for 30 minutes. After cooling, the solution was evaporated to dryness to afford the title sulfonyl chloride which was used directly in the next step.

### c) 8-Methoxy-3-methyl-2,3,4,5-tetrahydro-1H-3-benzazepine-7-sulfonyl fluoride

The sulfonyl chloride from part (b) was dissolved in acetonitrile (500 mL) and potassium fluoride (37 g, 625 mmol) and 18-crown-6 (1 crystal) added. The mixture was stirred for 18 hours, then quenched with cold aqueous sodium bicarbonate solution until the pH equalled 8. The mixture was extracted twice with ethyl acetate, washed with bicarbonate solution then brine, dried and evaporated to afford the sulfonyl fluoride (D1) (25 g).

### Description 2a

### 7-(4-Fluorobenzenesulfonyl)-8-methoxy-3-methyl-2,3,4,5-tetrahydro-1H-3-benzazepine (D2)

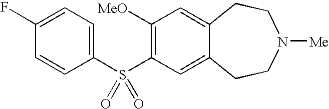

8-Methoxy-3-methyl-2,3,4,5-tetrahydro-1H-3-benzazepine-7-sulfonyl fluoride (25 g) was dissolved in dry tetrahydrofuran (250 mL) and 4-fluorophenylmagnesium bromide in tetrahydrofuran (2.5 equivalents) was added over 15 minutes with ice bath cooling, an exotherm only apparent during the first part of the addition. The resulting mixture was stirred overnight without cooling, then this solution was added over 10 minutes to a solution of sodium potassium tartrate tetrahydrate (250 g) in water (450 mL) with stirring. Diethyl ether was added (400 mL) and the organic layer separated, dried, evaporated, and the title product (D2) crystallised (17 g).

### Description 2b

### 7-(4-Fluorobenzenesulfonyl)-8-methoxy-3-methyl-2,3,4,5-tetrahydro-1H-3-benzazepine (D2)

### (i) 7-Methoxy-3-methyl-2,3,4,5-tetrahydro-1H-3-benzazepinium trifluoroacetate

Trifluoroacetic acid (2 mL) was added to a solution of 7-methoxy-3-methyl-2,3,4,5-tetrahydro-1H-3-benzazepine (5 g) in isopropyl acetate (20 mL), maintaining the temperature below 30° C. n-Heptane (20 mL) was added at 25° C., the mixture seeded and stirred at 20-25° C. to crystallize the product. The resulting solid was filtered, washed with n-heptane (10 mL) and dried under vacuum at 40-45° C. to give the title product as an off-white solid (6.4 g). Mp 91-92° C.; δ_{H} (400 MHz, DMSO) 2.84 (3H, s, NCH₃), 2.90-3.57 (8H, br m, CH₂CH₂), 3.73 (3H, s, OCH₃), 6.76 (1H, d, J=8Hz, ArH), 6.83 (1H, s, ArH), 7.13 (1H, d, J=8 Hz, ArH), 10.26 (1H, br s); MS (ES+) m/z 192 (MH⁺).

### (ii) 7-(4-Fluorobenzenesulfonyl)-8-methoxy-3-methyl-2,3,4,5-tetrahydro-1H-3-benzazepine (D2)

Trifluoromethanesulfonic acid (2.2 mL, 25 mmol) was added to a mixture of 7-methoxy-3-methyl-2,3,4,5-tetrahydro-1H-3-benzazepinium trifluoroacetate (5 g, 16.4 mmol), 4-fluorobenzenesulfonyl chloride (4.8 g, 25 mmol), and indium(III) chloride (0.36 g, 1.6 mmol) in trifluoroacetic acid (10 mL) at ambient temperature, under a nitrogen atmosphere. The resulting mixture was heated under reflux for 7 hours then cooled and diluted with dichloromethane (25 mL) followed by water (15 mL) whilst maintaining the temperature below 20° C. When the addition was complete, the pH was adjusted to 2 by the addition of 40% w/v aqueous sodium hydroxide (15 mL) and the phases separated. Water (10 mL) was added, followed by 10% w/v aqueous sodium hydroxide to adjust the pH to 10. The phases were separated and the organic phase washed with water (15 mL), dried (MgSO₄) and filtered. The filtrate was diluted with isopropyl acetate (35 mL) and concentrated under reduced pressure to a residual volume of 15 mL and stirred at ambient temperature to crystallise the product. The resulting slurry was stirred in an ice bath for 1 hour, then filtered and the cake was washed with 2:1 heptane:isopropyl acetate (10 mL), followed by drying the cake at 40° C. under vacuum to give the title product (D2) as a white solid (4.06 g). Mp 129-130° C.; δ_{H} (400 MHz, DMSO) 2.23 (3H, s, NCH₃), 2.44 (4H, m, CH₂ CH₂), 2.87 (4H, m, CH₂CH₂), 3.70 (3H, s, OCH₃), 6.97 (1H, s, ArH), 7.40 (2H, dd, J=9.0, 9.0 Hz, ArH), 7.70 (1H, s, ArH), 7.94 (2H, dd, J=9.0, 5.2 Hz, ArH); MS (ES+) m/z 350 (100%, MH⁺).

### Description 3

### Preparation of 7-[4-(4-chlorobenzyloxy)benzenesulfonyl]-8-methoxy-3-methyl-2,3,4,5-tetrahydro-1H-3-benzazepine (D3)

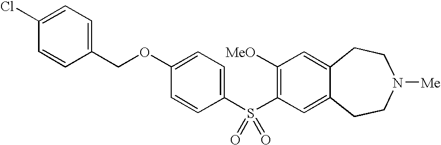

A solution of 4-chlorobenzyl alcohol (4.9 g, 34.4 mmol) in tetrahydrofuran (20 mL) was added drop-wise to a solution of potassium tert-butoxide (4.9 g, 43.2 mmol) in tetrahydrofuran (30 mL) maintaining the temperature below 25° C. The resulting mixture was stirred under nitrogen for 10 minutes then a solution of 7-(4-fluorobenzenesulfonyl)-8-methoxy-3-methyl-2,3,4,5-tetrahydro-1H-3-benzazepine (D2) (10 g, 28.6 mmol) in tetrahydrofuran (45 mL) was added drop-wise maintaining the temperature below 25° C. and the mixture stirred for 1.75 hours. 10% w/v Aqueous ammonium chloride (50 mL) was added and the mixture stirred for 5 minutes. The phases were separated, water (70 mL) was added to the organic phase and the mixture stirred at 15-25° C. for 1.5 hours. The resulting solid was filtered, the cake washed with water (20 mL) and dried at 50° C. under vacuum to yield the title product (D3) as a white solid (10.99 g). Mp 120-122° C.; δ_{H} (400 MHz, DMSO) 2.25 (3H, s, NCH₃), 2.46 (4H, m, CH₂CH₂), 2.88 (4H, m, CH₂CH₂), 3.70 (3H, s, OCH₃), 5.19 (2H, s, ArCH₂), 6.95 (1H, s, ArH), 7.16 (2H, d, J=7.0 Hz, ArH), 7.46 (4H, m, ArH), 7.68 (1H, s, ArH), 7.81 (2H, d, J=7.0 Hz, ArH); MS (ES+) m/z 474 (MH⁺), 472 (MH⁺, 100%) 192.

### EXAMPLE 1

### Preparation of 7-[4-(4-chlorobenzyloxy)benzenesulfonyl]-8-methoxy-3-methyl-2,3,4,5-tetrahydro-1H-3-benzazepinium maleate (E1)

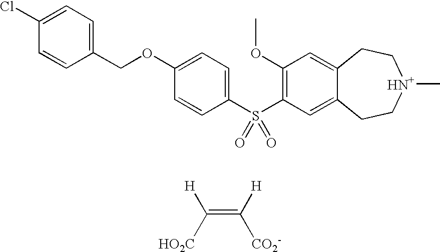

A solution of maleic acid (27.1 g, 233.4 mmol) in ethanol (100 mL) was added portionwise to a boiling solution of 7-[4-(4-chlorobenzyloxy)-benzenesulfonyl]-8-methoxy-3-methyl-2,3,4,5-tetrahydro-1H-3-benzazepine (D3) (100.1 g, 212.0 mmol) in ethanol (1.05 L) and the resulting solution allowed to stir for 10 minutes and return to reflux. The solution was cooled to 75° C., seeded with maleate salt (100.8 mg) then cooled to ambient temperature. The resulting slurry was stirred at ambient temperature for 2 hours and filtered; the cake was washed with ethanol (300 mL) and dried under vacuum at 60° C. to yield the title product (E1) as a white solid (122.4 g). Mp 170-172° C.; δ_{H} (400 MHz, DMSO) 2.81 (3H, s, NCH₃), 3.10 (4H, br s, CH₂CH₂), 3.34 (4H, br s, CH₂CH₂), 3.72 (3H, s, OCH₃), 5.18 ArCH₂), 6.02 (2H, s, —CH═CH—), 7.07 (1H, s, ArH), 7.17 (2H, d, J=7, ArH), 7.46 (4H, m, ArH), 7.80 (2H, d, J=7, ArH), 7.82 (1H, s, ArH), 9.0-10.0 (1H, br s); MS (ES+) m/z 474 (MH⁺), 472 (MH⁺, 100%) 192.

TABLE 1
X-Ray powder diffraction (XRPD) angles and d spacings
for 7-[4-(4-Chlorobenzyl-oxy)benzenesulfonyl]-8-methoxy-3-
methyl-2,3,4,5-tetrahydro-1H-3-benzazepinium maleate.
Peaks with relative intensities greater than 5% are recorded.

| Pos.[°2 Th.] | d-spacing[Å] |
|---|---|
| 5.9 | 15.0 |
| 9.6 | 9.2 |
| 10.0 | 8.8 |
| 10.2 | 8.6 |
| 11.3 | 7.8 |
| 11.5 | 7.7 |
| 14.8 | 6.0 |
| 15.5 | 5.0 |
| 16.2 | 5.5 |
| 16.9 | 5.2 |
| 17.2 | 5.1 |
| 18.2 | 4.9 |
| 18.8 | 4.7 |
| 19.5 | 4.5 |
| 19.7 | 4.5 |
| 20.0 | 4.4 |
| 20.5 | 4.3 |
| 21.3 | 4.2 |
| 21.8 | 4.1 |
| 22.0 | 4.0 |
| 23.3 | 3.8 |
| 24.0 | 3.7 |
| 24.6 | 3.6 |
| 24.8 | 3.6 |
| 25.0 | 3.6 |
| 25.5 | 3.5 |
| 25.9 | 3.4 |
| 26.1 | 3.4 |
| 26.9 | 3.3 |
| 27.1 | 3.3 |
| 27.4 | 3.3 |
| 27.9 | 3.2 |
| 28.1 | 3.2 |
| 28.6 | 3.1 |
| 28.8 | 3.1 |
| 29.7 | 3.0 |
| 30.4 | 2.9 |
| 33.7 | 2.7 |
| 35.7 | 2.5 |

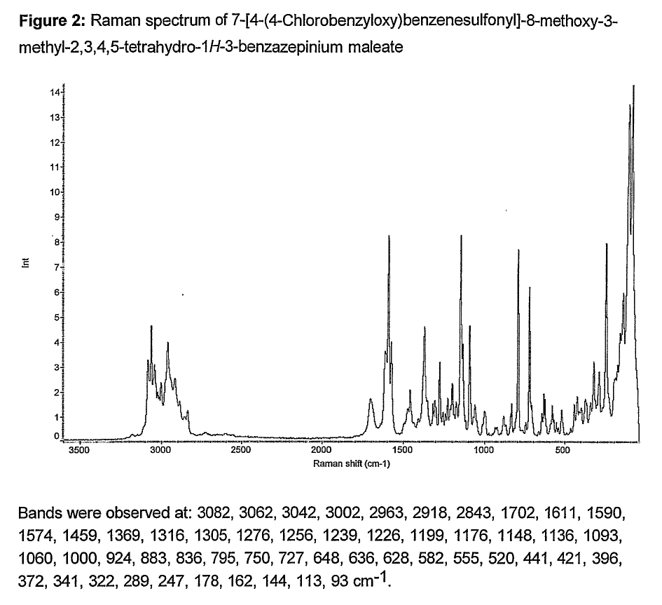

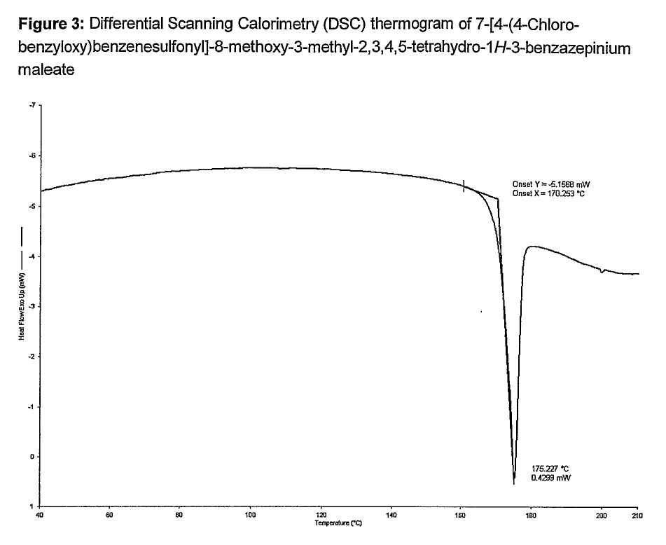

Data obtained for the maleate are shown in FIGS. 1-3 and Table 1.

### EXAMPLE 2

### Preparation of 7-[4-(4-Chlorobenzyloxy)benzenesulfonyl]-8-methoxy-3-methyl-2,3,4,5-tetrahydro-1H-3-benzazepinium p-toluenesulfonate (E2)

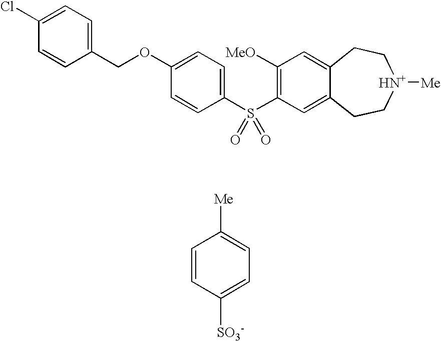

A solution of para-toluenesulfonic acid (105 mg, 0.55 mmol) in acetone (1 mL) was added dropwise to a solution of 7-[4-(4-chlorobenzyloxy)-benzenesulfonyl]-8-methoxy-3-methyl-2,3,4,5-tetrahydro-1H-3-benzazepine (D3) (255 mg, 0.54 mmol) in acetone (1.5 mL) at 50° C. The resulting solution was stirred at 50° C. for 30 minutes then cooled to ambient temperature and stirred for 1 hour. The resulting slurry was filtered, the filter cake washed with acetone (2.5 mL) and dried under vacuum at 45° C. to yield the title compound (E2) as a white solid (329 mg). Mp 190-192° C.; δ_{H} (400 MHz, DMSO) 2.35 (3H, s, ArCH₃), 2.90 (3H, s, NCH₃), 3.09-3.19 (6H, br m, CH₂CH₂), 3.65 (2H, br s, CH₂CH₂), 3.79 (3H, s OCH₃), 5.26 (2H, s, ArCH₂), 7.14-7.18 (3H, m, ArH), 7.22-7.25 (2H, m, ArH), 7.50-7.55 (6H, m, ArH), 7.86-7.89 (3H, m, ArH), 9.73 (1H, br s). MS (ES+) m/z 474 (MH⁺), 472 (MH⁺, 100%), 225, 192.

TABLE 2
XRPD angles and d spacings for 7-[4-(4-Chlorobenzyloxy)
benzenesulfonyl]-8-methoxy-3-methyl-2,3,4,5-tetrahydro-1H-
3-benzazepinium tosylate. Peaks with relative intensities
greater than 5% are recorded.

| Pos.[°2 Th.] | d-spacing[Å] |
|---|---|
| 5.7 | 15.5 |
| 6.4 | 13.8 |
| 9.6 | 9.2 |
| 11.5 | 7.7 |
| 12.1 | 7.3 |
| 13.8 | 6.4 |
| 14.1 | 6.3 |
| 14.5 | 6.1 |
| 15.8 | 5.6 |
| 16.5 | 5.4 |
| 17.2 | 5.2 |
| 18.7 | 4.7 |
| 19.5 | 4.5 |
| 19.9 | 4.5 |
| 20.4 | 4.4 |
| 21.0 | 4.2 |
| 21.3 | 4.2 |
| 21.8 | 4.1 |
| 22.2 | 4.0 |
| 22.8 | 3.9 |
| 23.4 | 3.8 |
| 23.8 | 3.7 |
| 24.5 | 3.6 |
| 25.1 | 3.6 |
| 26.0 | 3.4 |
| 27.3 | 3.3 |
| 28.3 | 3.2 |
| 29.1 | 3.1 |
| 30.5 | 2.9 |
| 33.4 | 2.7 |
| 34.4 | 2.6 |

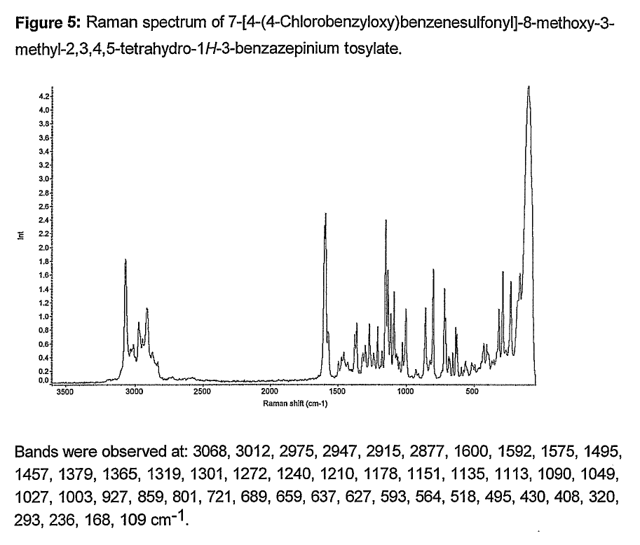

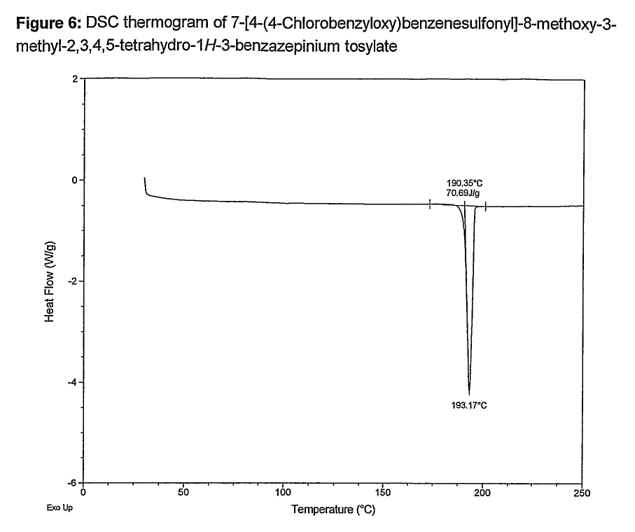

Data obtained for the tosylate are shown in FIGS. 4-6 and Table 2.

### EXAMPLE 3

### Preparation of 7-[4-(4-chlorobenzyloxy)benzenesulfonyl]-8-methoxy-3-methyl-2,3,4,5-tetrahydro-1H-3-benzazepinium maleate, dehydrate (E3)

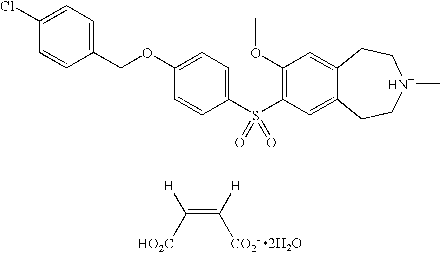

7-[4-(4-Chlorobenzyloxy)benzenesulfonyl]-8-methoxy-3-methyl-2,3,4,5-tetrahydro-1H-3-benzazepinium maleate (E1) (20.7 g) was stirred in a mixture of ethanol (15 mL) and water (135 mL) and heated to 70° C. The solution was cooled to ambient temperature, with stirring. The resulting slurry was stirred at ambient temperature for 2 hours and filtered; the cake was washed with 9:1 water:ethanol (100 mL) and dried under vacuum at 50° C. to yield the title product (E3) as a white solid (21.5 g). Mp 90-98° C.; Water content by Karl Fisher titration 5.9%; δ_{H} (400 MHz, DMSO) 2.83 (3H, s, NCH₃), 3.12 (4H, br s, CH₂CH₂), 3.33 (4H, br s, CH₂CH₂), 3.74 (3H, s, OCH₃), 5.21 (2H, s, ArCH₂), 6.04 (2H, s, —CH═CH—), 7.09 (1H, s, ArH), 7.19 (2H, d, J=7, ArH), 7.47 (4H, m, ArH), 7.82 (2H, d, J=7, ArH), 7.84 (1H, s, ArH), 9.0-10.0 (1H, br s); MS (ES+) m/z 474 (MH⁺), 472 (MH⁺, 100%), 192.

TABLE 3
XRPD angles and d spacings for 7-[4-(4-Chlorobenzyloxy)
benzenesulfonyl]-8-methoxy-3-methyl-2,3,4,5-tetrahydro-
1H-3-benzazepinium maleate, dihydrate Peaks with relative
intensities greater than 5% are recorded.

| Pos.[°2 Th.] | d-spacing[Å] |
|---|---|
| 6.8 | 13.0 |
| 9.7 | 9.1 |
| 9.8 | 9.0 |
| 10.1 | 8.7 |
| 12.3 | 7.2 |
| 13.6 | 6.5 |
| 13.8 | 6.4 |
| 15.5 | 5.7 |
| 15.7 | 5.6 |
| 16.5 | 5.4 |
| 18.3 | 4.8 |
| 19.4 | 4.6 |
| 19.6 | 4.5 |
| 19.8 | 4.5 |
| 20.3 | 4.4 |
| 20.4 | 4.4 |
| 20.6 | 4.3 |
| 20.9 | 4.3 |
| 21.1 | 4.2 |
| 21.8 | 4.1 |
| 22.7 | 3.9 |
| 23.0 | 3.9 |
| 24.4 | 3.6 |
| 24.8 | 3.6 |
| 25.4 | 3.5 |
| 26.5 | 3.4 |
| 26.8 | 3.3 |
| 27.3 | 3.3 |
| 27.6 | 3.2 |
| 27.7 | 3.2 |
| 27.8 | 3.2 |
| 28.3 | 3.2 |
| 28.8 | 3.1 |
| 28.8 | 3.1 |
| 29.6 | 3.0 |
| 29.9 | 3.0 |
| 30.0 | 3.0 |
| 30.6 | 2.9 |
| 30.7 | 2.9 |
| 31.3 | 2.9 |
| 31.7 | 2.8 |
| 31.8 | 2.8 |
| 32.8 | 2.7 |
| 33.2 | 2.7 |
| 33.6 | 2.7 |
| 34.1 | 2.6 |
| 35.1 | 2.6 |
| 37.0 | 2.4 |
| 37.8 | 2.4 |
| 39.6 | 2.3 |

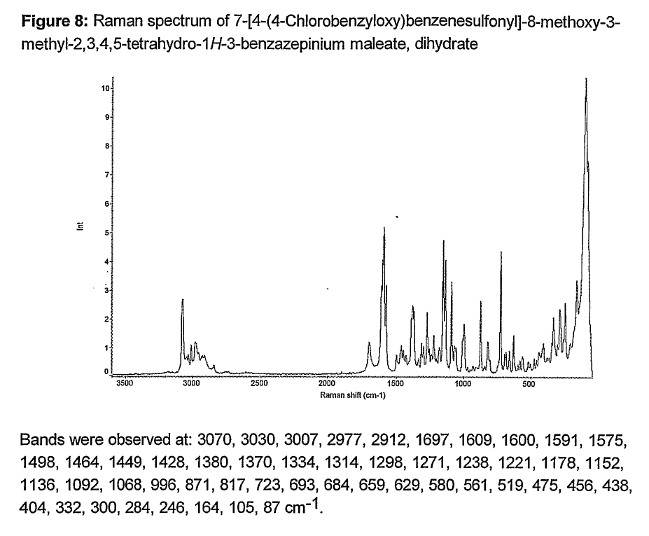

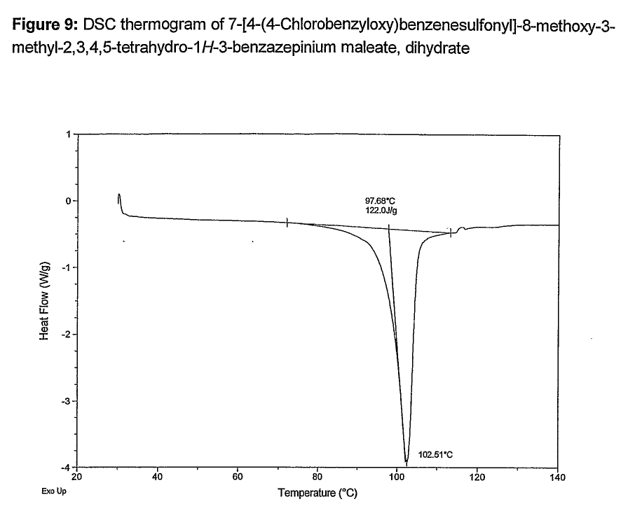

Data obtained for the maleate, dihydrate are shown in FIGS. 7-9 and Table 3.

### Example 4

### Preparation of 7-[4-(4-chlorobenzyloxy)benzenesulfonyl]-8-methoxy-3-methyl-2,3,4,5-tetrahydro-1H-3-benzazepinium maleate, acetic acid solvate (E4)

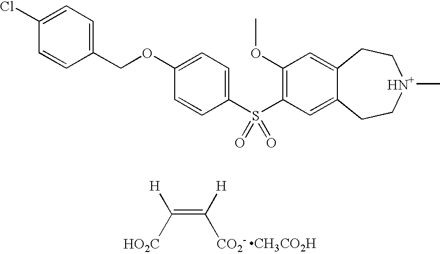

7-[4-(4-Chlorobenzyloxy)benzenesulfonyl]-8-methoxy-3-methyl-2,3,4,5-tetrahydro-1H-3-benzazepinium maleate (E1) (12 g) was stirred in acetic acid (10 mL) for 72 hours. The resulting product was filtered under suction for 30 minutes and the cake left to stand at ambient temperature and pressure for 18 hours to yield the title product (E4) as a white solid (13.2 g). Mp 96-98° C.; δ_{H} (400 MHz, DMSO) 1.92 (3H, s, CH₃CO₂H), 2.83 (3H, s, NCH₃), 3.12 (4H, br s, CH₂CH₂), 3.32 (4H, br s, CH₂CH₂), 3.73 (3H, s, OCH₃), 5.20 (2H, s, ArCH₂), 6.03 (2H, s, —CH═CH—), 7.09 (1H, s, ArH), 7.18 (2H, d, J=8, ArH), 7.47 (4H, m, ArH), 7.82 (3H, m, ArH); MS (ES+) m/z 474 (MH⁺), 472 (MH⁺, 100%), 192.

TABLE 4
XRPD angles and d spacings for 7-[4-(4-Chlorobenzyloxy)
benzenesulfonyl]-8-methoxy-3-methyl-2,3,4,5-tetrahydro-
1H-3-benzazepinium maleate, acetic acid solvate

| Pos.[°2 Th.] | d-spacing[Å] |
|---|---|
| 6.3 | 14.1 |
| 9.5 | 13.7 |
| 10.2 | 8.7 |
| 11.8 | 7.5 |
| 12.6 | 7.0 |
| 13.9 | 6.4 |
| 14.3 | 6.2 |
| 15.0 | 5.9 |
| 15.7 | 5.6 |
| 16.3 | 5.4 |
| 16.8 | 5.3 |
| 17.2 | 5.1 |
| 18.7 | 4.7 |
| 18.9 | 4.7 |
| 19.1 | 4.7 |
| 19.2 | 4.6 |
| 19.5 | 4.5 |
| 19.7 | 4.5 |
| 20.6 | 4.3 |
| 21.5 | 4.1 |
| 22.5 | 3.9 |
| 22.8 | 3.9 |
| 23.0 | 3.9 |
| 23.4 | 3.8 |
| 23.8 | 3.7 |
| 24.3 | 3.7 |
| 24.9 | 3.6 |
| 25.1 | 3.5 |
| 25.4 | 3.5 |
| 26.2 | 3.4 |
| 26.5 | 3.4 |
| 26.9 | 3.3 |
| 27.2 | 3.3 |
| 27.5 | 3.2 |
| 27.9 | 3.2 |
| 28.4 | 3.1 |
| 28.9 | 3.1 |
| 29.7 | 3.0 |
| 30.0 | 3.0 |
| 31.0 | 2.9 |
| 31.7 | 2.8 |
| 32.7 | 2.7 |
| 33.5 | 2.7 |
| 34.0 | 2.6 |
| 35.2 | 2.6 |
| 35.7 | 2.5 |
| 37.2 | 2.4 |
| 37.9 | 2.4 |
| 38.5 | 2.3 |
| 39.1 | 2.3 |

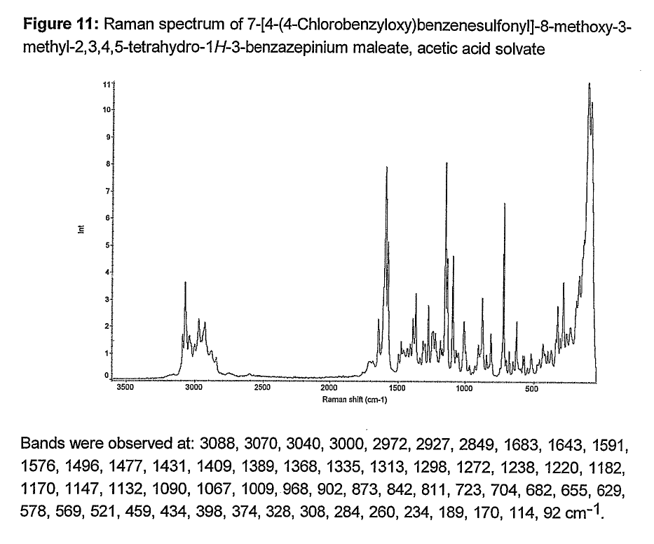

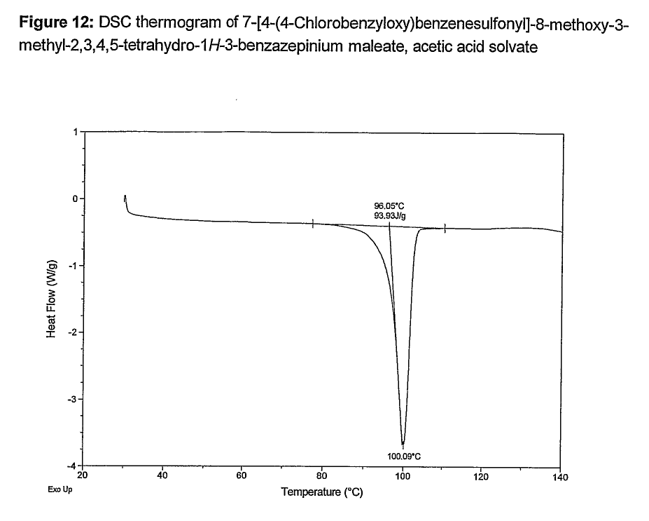

Data obtained for the maleate, acetic acid solvate are shown in FIGS. 10-12 and Table 4.

X-Ray Powder Diffraction

X-Ray Powder Diffraction (XRPD) analysis was performed on a Phillips X'pert Pro powder diffractometer, using an X'Celerator detector. The acquisition conditions were; radiation: Cu K_{α}, generator tension: 40 kV, generator current: 45 mA, start angle: 2.0 °2θ, end angle: 40.0 °2θ, step size: 0.0167 °2θ, time per step: 31.75 seconds. The samples of maleate and tosylate were prepared using backfill technique. The samples of maleate, dihydrate and acetic acid solvate were prepared using silicon wafer technique.

Raman Spectroscopy

Raman spectra were recorded in an NMR tube using a Nicolet 960 E.S.P. FT-Raman spectrometer, at 4 cm⁻¹ resolution with excitation from a Nd:VO₄ laser (1064 nm) with a power output of 400 mW. An absolute threshold of 0.5 and sensitivity of 65% were applied for the purpose of peak selection.

Differential Scanning Calorimetry (DSC)

DSC thermograms for the maleate and tosylate were recorded using a Perkin Elmer Diamond DSC. DSC thermograms for the maleate, dihydrate and acetic acid solvate were recorded using a Thermal Analysis DSC Q1000. The sample was heated at 10° C. min⁻¹ in an open pan.

All publications, including but not limited to patents and patent applications, cited in this specification are herein incorporated by reference as if each individual publication were specifically and individually indicated to be incorporated by reference herein as though fully set forth.

1. One or more chemical entities selected from 7-[4-(4-chlorobenzyloxy)benzenesulfonyl]-8-methoxy-3-methyl-2,3,4,5-tetrahydro-1H-3-benzazepinium maleate and a pharmaceutically acceptable solvate thereof.

2. One or more chemical entities selected from 7-[4-(4-chlorobenzyloxy)-benzenesulfonyl]-8-methoxy-3-methyl-2,3,4,5-tetrahydro-1H-3-benzazepinium tosylate and a pharmaceutically acceptable solvate thereof.

3. A chemical entity according to claim 1 wherein the ratio of 7-[4-(4-chlorobenzyloxy)benzenesulfonyl]-8-methoxy-3-methyl-2,3,4,5-tetrahydro-1H-3-benzazepine to maleic acid (by mole) is 1:1.

4. A chemical entity according to claim 2 wherein the ratio of 7-[4-(4-chlorobenzyloxy)benzenesulfonyl]-8-methoxy-3-methyl-2,3,4,5-tetrahydro-1H-3-benzazepine to p-toluenesulfonic acid (by mole) is 1:1.

5. A chemical entity according to claim 1 in crystalline form.

6. A pharmaceutical composition comprising a chemical entity as claimed in claim 1 and a pharmaceutically acceptable carrier.

7-9. (canceled)

10. A method of treatment of a psychotic disorder in mammals including humans, which comprises administering to a mammal in need thereof an effective amount of a chemical entity according to claim 1.

11. A chemical entity according to claim 2 in crystalline form.

12. A pharmaceutical composition comprising a chemical entity as claimed in claim 2 and a pharmaceutically acceptable carrier.

13. A method of treatment of a psychotic disorder in mammals including humans, which comprises administering to a mammal in need thereof an effective amount of a chemical entity according to claim 2.
